(19) United States
(12) Patent Application Publication
(10) Pub. No.: US 2024/0423734 A1
(43) Pub. Date: Dec. 26, 2024

(54) SYSTEM AND METHOD TO RESTRICT RANGE OF MOTION OF ROBOTIC SURGICAL SYSTEM

(71) Applicant: Cilag GmbH International, Zug (CH)

(72) Inventors: Tracey A. Morley, Sunnyvale, CA (US); Charles J. Scheib, Loveland, OH (US); Mario F. Luces Rosado, Oakland, CA (US); Dillon R. Carey, Mountain View, CA (US); Michael K. Dermksian, Redwood City, CA (US); Matthew T. Hill, Norwood, OH (US)

(21) Appl. No.: 18/340,953

(22) Filed: Jun. 26, 2023

(51) Int. Cl. A61B 34/37; A61B 17/42; A61B 34/10; A61B 34/20

(52) U.S. Cl. CPC A61B 34/37; A61B 17/4241; A61B 34/10; A61B 34/20; A61B 2017/4225; A61B 2034/107; A61B 2034/2055; A61B 2034/302; A61B 2034/305

(57) ABSTRACT

A robotic surgical system includes a control console configured to generate a set of actuation instructions. A robotic arm is in communication with the control console and is configured to actuate an instrument based on the set of actuation instructions. The control console is configured to calculate an external zone of operation for the robotic arm based on patient tracking data, the patient tracking data indicating a position of at least one external anatomical structure of the patient. The control console is further configured to calculate an instrument zone of operation for at least a portion of the instrument based on anatomical data received by the control console or instrument data received by the control console.

### BACKGROUND

A variety of medical instruments may be used in procedures conducted by a medical professional operator, as well as applications in robotically assisted surgeries. In the case of robotically assisted surgery, the clinician may operate a master controller to remotely control the motion of such medical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the medical instrument. In some scenarios, a servo motor moves a manipulator supporting the medical instrument based on the clinician's manipulation of the hand input devices. During the medical procedure, the clinician may employ, via a robotic system, a variety of medical instruments including an ultrasonic blade, a surgical stapler, a tissue grasper, a needle driver, an electrosurgical cautery probes, etc. Each of these structures performs functions for the clinician, for example, cutting tissue, coagulating tissue, holding or driving a needle, grasping a blood vessel, dissecting tissue, or cauterizing tissue.

Examples of robotic systems are described in U.S. Pat. No. 9,763,741, entitled “System for Robotic-Assisted Endolumenal Surgery and Related Methods,” issued Sep. 19, 2017, the disclosure of which is incorporated by reference herein, in its entirety; U.S. Pat. No. 10,464,209, entitled “Robotic System with Indication of Boundary for Robotic Arm,” issued Nov. 5, 2019, the disclosure of which is incorporated by reference herein, in its entirety; U.S. Pat. No. 10,667,875, entitled “Systems and Techniques for Providing Multiple Perspectives During Medical Procedures,” issued Jun. 2, 2020, the disclosure of which is incorporated by reference herein, in its entirety; U.S. Pat. No. 10,765,303, entitled “System and Method for Driving Medical Instrument,” issued Sep. 8, 2020, the disclosure of which is incorporated by reference herein, in its entirety; U.S. Pat. No. 10,827,913, entitled “Systems and Methods for Displaying Estimated Location of Instrument,” issued Nov. 10, 2020, the disclosure of which is incorporated by reference herein, in its entirety; U.S. Pat. No. 10,881,280, entitled “Manually and Robotically Controllable Medical Instruments,” issued Jan. 5, 2021, the disclosure of which is incorporated by reference herein, in its entirety; U.S. Pat. No. 10,898,277, entitled “Systems and Methods for Registration of Location Sensors,” issued Jan. 26, 2012, the disclosure of which is incorporated by reference herein, in its entirety; and U.S. Pat. No. 11,058,493, entitled “Robotic System Configured for Navigation Path Tracing,” issued Jul. 13, 2021, the disclosure of which is incorporated by reference herein, in its entirety.

During a hysterectomy procedure, a colpotomy may be performed at the cervicovaginal junction. Such procedures may include the use of a uterine manipulator that includes a colpotomy cup or similar structure. Examples of instruments that may be used during a hysterectomy procedure are described in U.S. Pat. No. 9,743,955, entitled “Intracorporeal Transilluminator of Tissue Using LED Array,” issued Aug. 29, 2017; U.S. Pat. No. 9,788,859, entitled “Uterine Manipulators and Related Components and Methods,” issued Oct. 17, 2017; U.S. Pat. No. 10,639,072, entitled “Uterine Manipulator,” issued May 5, 2020; U.S. Pat. No. 11,090,082, entitled “Colpotomy Systems, Devices, and Methods with Rotational Cutting,” issued Aug. 17, 2021; and U.S. Pub. No. 2021/0100584, entitled “Uterine Manipulator,” published Apr. 8, 2021.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 depicts a perspective view of an example of a table-based robotic system that includes a control console and a plurality of robotic arms.

FIG. 2 depicts a perspective view of an example of a robotic arm, an example of a tool drive, and an example of an instrument, each configured for use with the table-based robotic system of FIG. 1.

FIG. 3 depicts a perspective view of an example of a uterine manipulator that may be operatively attached to the tool drive of FIG. 2.

FIG. 4 depicts a perspective view of a colpotomy cup of the uterine manipulator of FIG. 3.

FIG. 5 depicts a cross-sectional view of the colpotomy cup of FIG. 4, taken along line 5-5 of FIG. 4.

FIG. 6A depicts a mid-sagittal cross-sectional view of a vagina and uterus.

FIG. 6B depicts a mid-sagittal cross-sectional view of the vagina and uterus of FIG. 6A, with the shaft of the uterine manipulator instrument of FIG. 3 inserted through the vagina into the uterus, with a balloon of the uterine manipulator instrument in a deflated state, and with a sleeve of the uterine manipulator instrument in a proximal position.

FIG. 6C depicts a mid-sagittal cross-sectional view of the vagina and uterus of FIG. 6A, with the shaft of the uterine manipulator instrument of FIG. 3 inserted through the vagina into the uterus, with the balloon of the uterine manipulator instrument in an inflated state, and with the sleeve of the uterine manipulator instrument in the proximal position.

FIG. 6D depicts a mid-sagittal cross-sectional view of the vagina and uterus of FIG. 6A, with the shaft of the uterine manipulator instrument of FIG. 3 inserted through the vagina into the uterus, with the balloon of the uterine manipulator instrument in the inflated state, with the sleeve of the uterine manipulator instrument in a distal position such that the colpotomy cup of the sleeve is engaged with the cervix, and with a balloon of the sleeve in a deflated state.

FIG. 6E depicts a mid-sagittal cross-sectional view of the vagina and uterus of FIG. 6A, with the shaft of the uterine manipulator instrument of FIG. 3 inserted through the vagina into the uterus, with the balloon of the uterine manipulator instrument in the inflated state, with the sleeve of the uterine manipulator instrument in the distal position such that the colpotomy cup of the sleeve is engaged with the cervix, and with the balloon of the sleeve in an inflated state.

FIG. 7A depicts a partially exploded perspective view of an alternative colpotomy cup.

FIG. 7B depicts a perspective view of the colpotomy cup of FIG. 7A.

FIG. 8 depicts a mid-sagittal cross-sectional view of the vagina and uterus of FIG. 6A, with an example of an alternative uterine manipulator operatively engaged with the vagina and uterus and attached to the robotic arm and tool drive of FIG. 2, where movement of the uterine manipulator is restricted within an internal volume of operation.

FIG. 9 depicts a top plan view of a patient on a support table with legs of the patient in stirrups and a visual tracking system positioned to track the position of the legs of the patient to control movement of the robotic arm of FIG. 1 within an external volume of operation.

FIG. 10 depicts a flowchart of an example of an algorithm for the robotic system of FIG. 1 to determine the internal volume of operation of FIG. 8.

FIG. 11 depicts a flowchart of an example of an algorithm for the robotic system of FIG. 1 to determine the external volume of operation of FIG. 9.

FIG. 12 depicts a flowchart of an example of an algorithm for the robotic system of FIG. 1 to filter user input commands to limit movement of the robotic arm of FIG. 2 and the uterine manipulator of FIG. 8 within the internal volume of operation of FIG. 8 and the external volume of operation of FIG. 9.

FIG. 13 depicts a perspective view of a proximal end of an example of an alternative uterine manipulator having a spatial tracking structure.

FIG. 14A depicts a mid-sagittal cross-sectional view of the vagina and uterus of FIG. 6A, with the uterine manipulator of FIG. 13 operatively engaged with the vagina and uterus and the visual tracking system of FIG. 9 tracking the position of the uterine manipulator.

FIG. 14B depicts a mid-sagittal cross-sectional view of the vagina and uterus of FIG. 6A, with the uterine manipulator of FIG. 13 operatively engaged with the vagina and uterus and the visual tracking system of FIG. 9 tracking the position of the uterine manipulator, where the robotic arm and tool drive of FIG. 2 are actuated toward a base of the uterine manipulator.

FIG. 14C depicts a mid-sagittal cross-sectional view of the vagina and uterus of FIG. 6A, with the uterine manipulator of FIG. 13 operatively engaged with the vagina and uterus and the visual tracking system of FIG. 9 tracking the position of the uterine manipulator, where the robotic arm and tool drive of FIG. 2 are operatively coupled with the base of the uterine manipulator.

FIG. 15 depicts a flowchart of an example of an algorithm for the robotic system of FIG. 1 to register the position of the uterine manipulator of FIG. 13 for purposes of coupling the robotic arm and tool driver of FIG. 2 with the uterine manipulator.

FIG. 16A depicts a mid-sagittal cross-sectional view of the vagina and uterus of FIG. 6A, with a distal portion of an alternative illustrative uterine manipulator operatively engaged with the vagina and uterus and a proximal portion of the uterine manipulator attached to the robotic arm and tool drive of FIG. 2, where the proximal portion and the distal portion are decoupled from each other yet connected to each other via a mechanical alignment and coupling assembly.

FIG. 16B depicts a mid-sagittal cross-sectional view of the vagina and uterus of FIG. 6A, with a distal portion of an example of an illustrative uterine manipulator operatively engaged with the vagina and uterus and a proximal portion of the uterine manipulator attached to the robotic arm and tool drive of FIG. 2, where the mechanical alignment and coupling assembly of FIG. 16A couples the proximal portion and the distal portion together.

### DETAILED DESCRIPTION

### I. Overview of Example of Robotic Surgical System

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification. A. Example of Robotic System Table

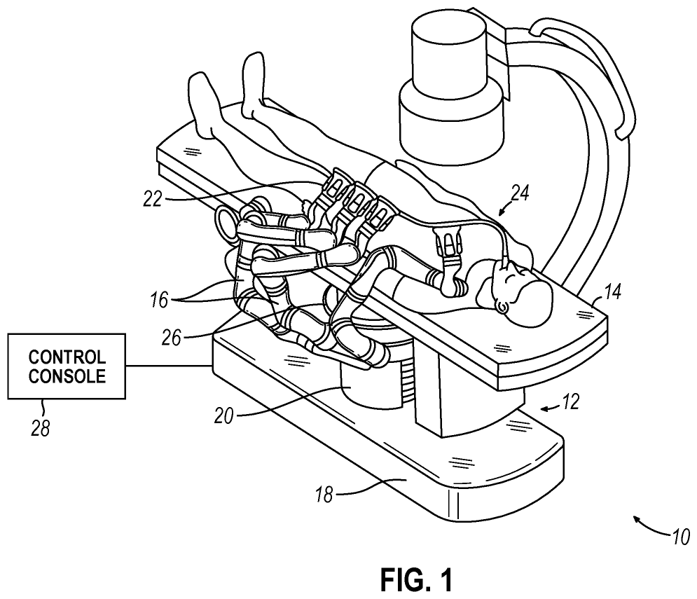

FIG. 1 illustrates an example of a robotic surgical system (10). Robotic surgical system (10) includes a support structure (12) for supporting a platform (14) (shown as a “table” or “bed”) over the floor and one or more robotic arms (16). Support structure (12) includes a base (18) and a column (20). Column (20) structurally supports platform (14) and provides a path for vertical translation of the carriages. In some versions, a table base may stow and store robotic arms (16) when not in use. Column (20) of the present example also includes a ring-shaped carriage (26), from which robotic arms (16) are based. A control console (28) is coupled with robotic surgical system (10).

Robotic arms (16) are shown as part of a table-mounted system, but in other configurations, robotic arms (16) may be mounted in a cart, ceiling or sidewall, or other suitable support surface. Robotic arms (16) are shown as extending from column (20) via carriage (26). However, robotic arms (16) may be coupled with robotic surgical system (10) using a variety of suitable structures. While robotic arms (16) are all shown as being positioned on one side of the patient in FIG. 1, other configurations may position robotic arms (16) on both sides of the patient, between the legs of the patient, and/or in any other suitable locations. Tool drivers (22) are positioned at distal ends of robotic arms (16) in the present example. Tool drivers (22) are operable to manipulate one or more instruments (24), as will be described in greater detail below.

### B. Example of a Robotic Arm, Tool Drive, and Tool

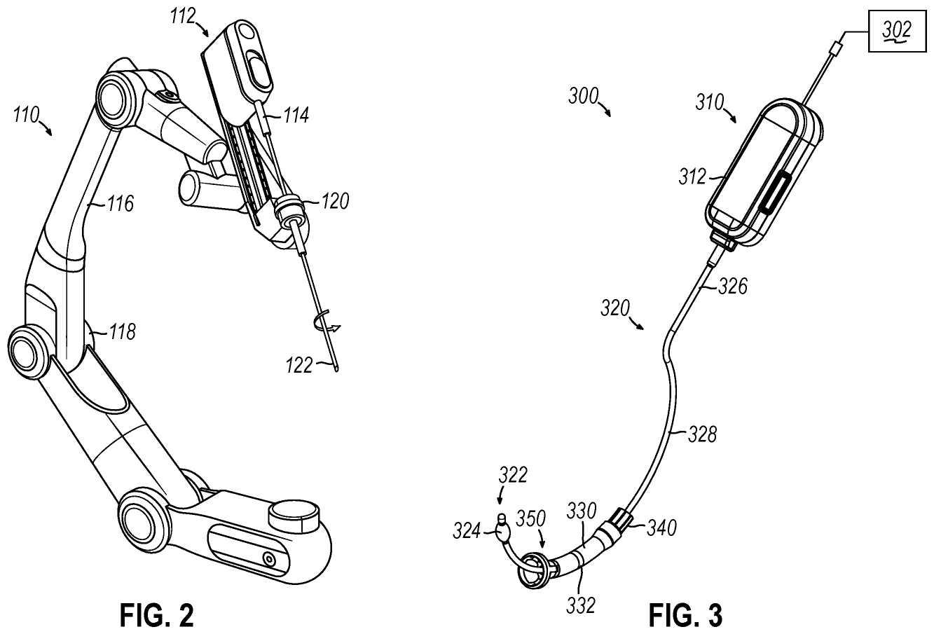

FIG. 2 shows an example of a robotic arm (110), a tool driver (112), and an instrument (114), which may be incorporated into robotic surgical system (10) in place of a robotic arm (16), a tool driver (22), and an instrument (24) that are shown in FIG. 1. Additional examples of robotic arms, a tool drivers, and instruments are shown and described in U.S. Pat. No. 10,166,082, entitled “System and Method for Controlling a Robotic Wrist,” issued Jan. 1, 2019, the disclosure of which is incorporated by reference herein, in its entirety.

As shown in FIG. 2, robotic arm (110) includes a plurality of links (116) and a plurality of joints (118) for actuating links (116) relative to one another. Tool driver (112) is attached to the distal end of robotic arm (110). Tool driver (112) includes a cannula (120) coupled to the end of tool driver (112), to receive and guide instrument (114). Instrument (114) may include an endoscope, a laparoscope, a stapler, graspers, an ultrasonic instrument, an RF electrosurgical instrument, or any other suitable kind of instrument. Instrument (114) is inserted into the patient via cannula (120). The distal end of instrument (114) includes an end effector (122). End effector (122) is configured to interact with the patient (e.g., providing visualization, stapling, grasping, ultrasonic cutting and/or sealing, electrosurgical cutting and/or sealing, etc.).

Joints (118) of robotic arm (110) may be actuated to selectively position and orient tool driver (112), which actuates the end effector (122) for robotic surgeries. Joints (118) may include various types, such as a pitch joint or a roll joint, which may substantially constrain the movement of the adjacent links (116) around certain axes relative to other links (116). Each joint (118) represents an independent degree of freedom available to robotic arm (110). A multitude of joints (118) result in a multitude of degrees of freedom, allowing for “redundant” degrees of freedom. Redundant degrees of freedom allow the robotic arms (110) to position their respective end effectors (122) at a specific position, orientation, and trajectory in space using different positions links (116) and angles of joints (118). This allows for the system to position and direct an instrument (114) from a desired point in space while allowing the clinician to move joints (118) into a clinically advantageous position away from the patient to create greater access, while avoiding collisions of robotic arms (110).

### II. Example of Robotically Controlled Uterine Manipulator

In some conventional hysterectomy procedures, a first clinician may serve in a role of forming incisions and performing other laparoscopic operations to remove the uterus of a patient, while a second clinician may serve in a role of manipulating the position and orientation of the uterus of the patient to facilitate the operations being performed by the first clinician. Such team-based procedures may require clear communication between the first clinician and the second clinician, with the first clinician instructing the second clinician on desired positioning and orientation of the uterus, and with the second clinician responding in a timely and accurate fashion. In some scenarios, such communications may break down or otherwise yield undesirable results, such as the second clinician not precisely positioning or orienting the uterus when and where the first clinician wishes. It may therefore be desirable to provide a robotic system that is capable of performing at least part of the role of the second clinician, such that the robotic system may at least partially control the position and orientation of the uterus based on the desire of the first clinician. Examples of how a robotic system may provide uterine manipulation are described in greater detail below. The following examples may be readily incorporated into robotic system (10) described herein; or in any other suitable robotic system as would be apparent to one skilled in the art in view of the teachings herein.

FIG. 3 shows an example of a uterine manipulator (300) that may be secured to a robotic arm (100) shown in FIG. 2 in replacement of instrument (114). Uterine manipulator (300) may be removably coupled with tool driver (112) and/or cannula (120), such that robotic arm (110) may selectively position and orient uterine manipulator (300) in relation to a patient by driving robotic arm (110). As best seen in FIG. 3, uterine manipulator (300) of the present example includes a tool driver interface (310), a shaft (320), a sleeve (330), a sleeve locking ring (340), and a colpotomy cup (350). Tool driver interface (310) includes a base (312). Base (312) is configured to removably couple with tool driver (112) of robotic arm (110) to thereby secure uterine manipulator (300) with robotic arm (110).

A linear portion (326) of shaft (320) that extends distally from base (312) may extend through cannula (120) when uterine manipulator (300) is coupled to robotic arm (110). In some instances, linear portion (326) of shaft (320) slidably extends through cannula (120); while in other instances linear portion (326) of shaft (320) may be temporarily secured to cannula (120). By way of example only, base (312) and tool driver (112) may include complementary bayonet fitting features, complementary threading, complementary snap-fit features, and/or any other suitable kinds of structures to provide a removable coupling. Shaft (320) is configured to couple with a pressurized fluid source (302). Pressurized fluid source (302) may contain pressurized air, pressurized saline, or any other suitable kind of pressurized fluid. The pressurized fluid may be used to selectively inflate balloons (324, 332), which will be described in greater detail below.

Shaft (320) of the present example extends distally from base (312). Shaft (320) includes proximal linear portion (326) and distal curved portion (328). In some versions, shaft (320) is rigid. In some other versions, shaft (320) is flexible yet resiliently biased to assume the curved configuration shown. Any suitable biocompatible material(s) may be used to form shaft (320), including but not limited to metallic materials, plastic materials, and combinations thereof. An inflatable balloon (324) is positioned near distal end (322) of shaft (320). Balloon (324) may be formed of an extensible material or a non-extensible material. The interior of shaft (320) includes one or more lumen(s) that are configured to communicate pressurized fluid from pressurized fluid source (302) to balloon (324). While balloon (324) is positioned near distal end (322) of shaft (320) in the present example, other versions may include a different kind of expandable member. By way of example only, an alternative expandable member may include a mechanically expandable component such as an expandable mesh structure, an expanding umbrella-like structure, or any other suitable kind of expandable structure or assembly. In some versions, distal end (322) of shaft (320) may also include an illuminating element (e.g., one or more LEDs, a lens illuminated by one or more optical fibers, etc.). In such versions, one or more wires, optical fibers, and/or other components may extend along the length of shaft (320) to couple with a source of electrical power, a source of light, etc.

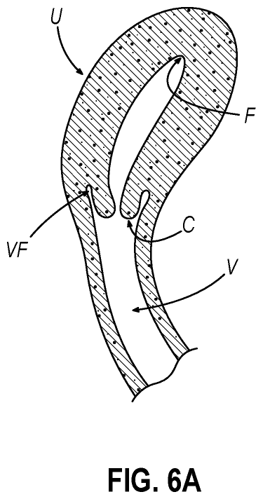

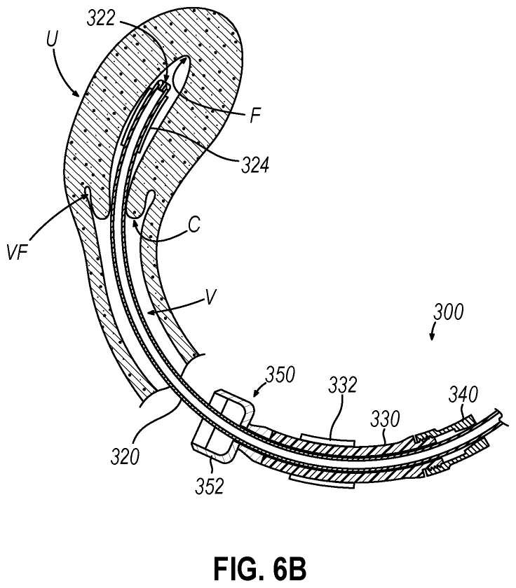

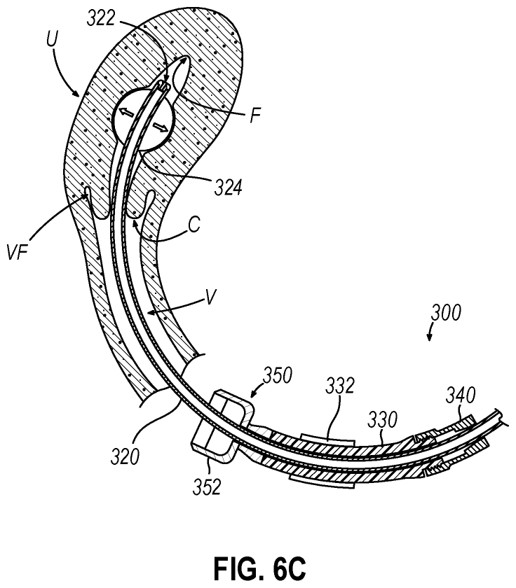

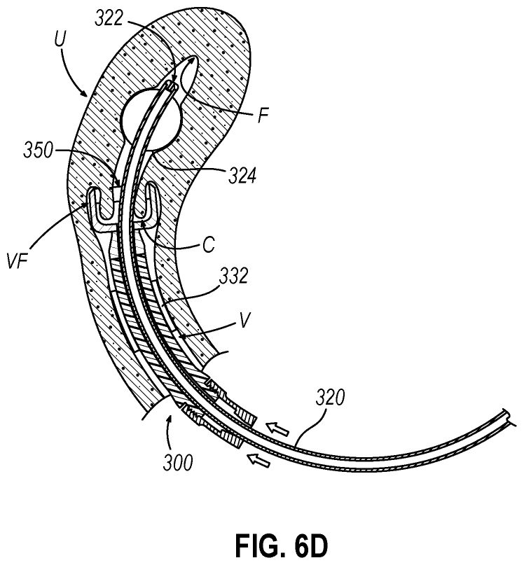

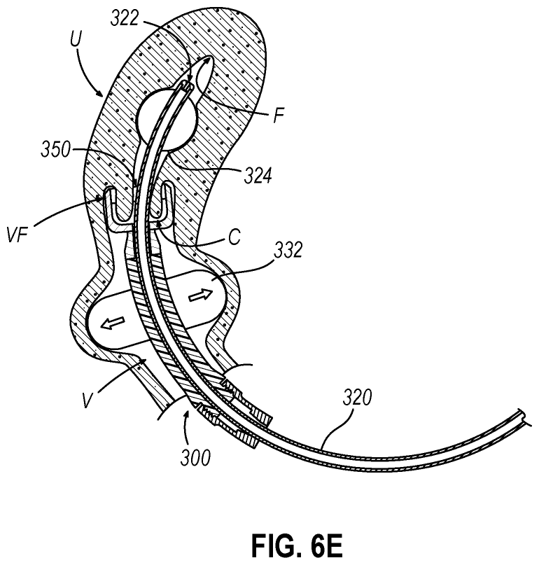

Sleeve (330) is slidably coupled to distal curved portion (328) of shaft (320), such that sleeve (330) may slide along shaft (320) from a proximal position (FIGS. 6B-6C) to any number of distal positions (FIGS. 3, 6D-6E). Sleeve (330) is generally cylindraceous and rigid; and extends along a curved axis such that the curved profile complements the curved profile of curved portion (328) of shaft (320). Sleeve (330) may be formed of plastic, metal, and/or any other suitable biocompatible material(s), including combinations of materials. Locking ring (340) is rotatably secured to the proximal end of sleeve (330), while colpotomy cup (350) is fixedly secured to the distal end of sleeve (330). An inflatable balloon (332) is positioned along sleeve (330), between locking ring (340) and colpotomy cup (350). Balloon (332) may be formed of an extensible material or a non-extensible material. The interior of sleeve (330) includes one or more lumen(s) that are configured to communicate pressurized fluid from pressurized fluid source (302) to balloon (332). Such a lumen or lumens may be coupled with pressurized fluid source (302) via a flexible tube (not shown). In some versions, one or more lumens or tubes within shaft (320) provide at least part of the fluid pathway between balloon (332) and pressurized fluid source (302).

Locking ring (340) is operable to selectively secure the position of sleeve (330) along the length of shaft (320). For instance, locking ring (340) may be rotated to a first angular position relative to sleeve (330) to provide an unlocked state where sleeve (330) may be freely translated along shaft (320). Locking ring (340) may then be rotated to a second angular position relative to sleeve (330) to provide a locked state where the position of sleeve (330) along shaft (320) is secured until locking ring (340) is rotated back to the first angular position. By way of example only, locking ring (340) may include one or more frictional braking structures that selectively engage shaft (320) to thereby provide the locked state. Alternatively, locking ring (340) may selectively engage shaft (320) in any other suitable fashion.

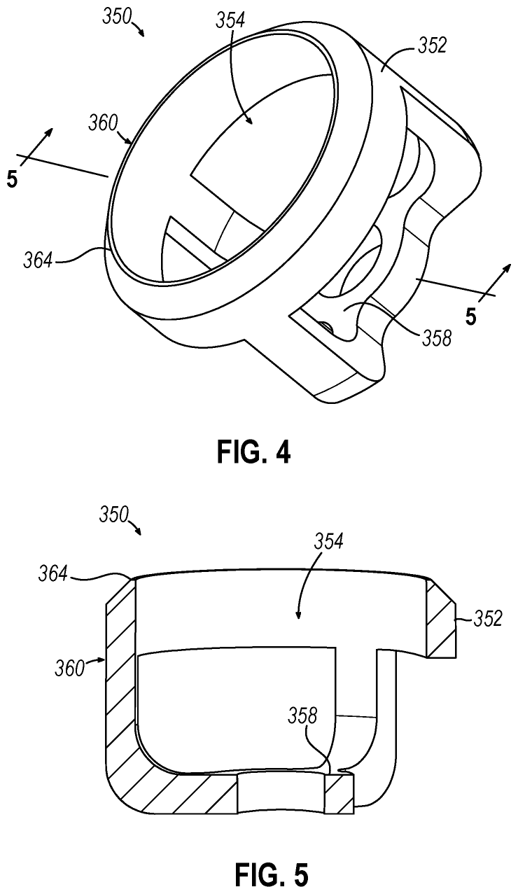

FIGS. 4-5 show colpotomy cup (350) in greater detail. As shown, colpotomy cup (350) of the present example includes a body (352) defining an interior space (354). Body (352) further includes a floor (358) at the bottom of interior space (354) and an open distal end (360). A plurality of lateral openings (356) are in communication with interior space (354). Distal end (360) includes a distally presented annular edge (364). Colpotomy cup (350) may be formed of plastic, metal, and/or any other suitable biocompatible material(s), including combinations of materials.

FIGS. 6A-6E show an example of a procedure in which uterine manipulator (300) is used. As shown in FIG. 6A, the anatomical context in which uterine manipulator (300) is used includes a vagina (V) and uterus (U) of a patient. As shown in FIG. 6B, distal end (322) of shaft (320) is inserted through the vagina (V) and into the uterus (U) via the cervix (C), while sleeve (330) is in a proximal position along shaft (320). Balloon (324) is in a deflated state during this stage of insertion. In some versions, uterine manipulator (300) is fully decoupled from robotic arm (110) during the process leading up to the stage shown in FIG. 6B, such that uterine manipulator (300) is advanced to this state manually by a human operator grasping a proximal portion of uterine manipulator (300) (e.g., grasping a proximal portion of shaft (320), grasping base (312), and/or grasping some other part of uterine manipulator (300)). In such scenarios, uterine manipulator (300) may be coupled with robotic arm (110) after reaching the stage shown in FIG. 6B.

In some other versions, uterine manipulator (300) is already coupled with robotic arm (110) before reaching the stage shown in FIG. 6B; and robotic arm (110) is used to guide and drive uterine manipulator (300) to the position shown in FIG. 6B. As yet another variation, some versions may allow a human operator to guide and drive uterine manipulator (300) to the position shown in FIG. 6B while uterine manipulator (300) is coupled with robotic arm (110), such that robotic arm (110) does not restrict manual movement of uterine manipulator (300) leading up to the stage shown in FIG. 6B.

Regardless of the stage at which uterine manipulator (300) is coupled with robotic arm (110), robotic arm (110) may be positioned in various suitable ways relative to the patient while uterine manipulator (300) is inserted in the patient. In some scenarios, robotic arm (110) crosses over the top of one of the patient's legs from the side, to assist in positioning uterine manipulator (300). In some other scenarios (e.g., when the patient's legs are supported by stirrups), robotic arm (110) crosses under the bottom of one of the patient's legs from the side, to assist in positioning uterine manipulator (300). In still other scenarios, robotic arm (110) is positioned between the patient's legs from underneath, such that robotic arm (110) does not cross over or under either of the patient's legs. Alternatively, robotic arm (110) may have any other suitable spatial and positional relationship with respect to the patient.

In the present example, uterine manipulator (300) is advanced distally until distal end (322) of shaft (320) reaches the fundus (F) of the uterus (U). The operator may determine that distal end (322) has reached the fundus (F) via tactile feedback (e.g., such that the operator can feel sudden resistance to further advancement of shaft (320)). In some cases where distal end (322) contacts the fundus (F), distal end (322) may remain in contact with fundus (F) throughout the rest of the procedure shown in FIGS. 6B-6E. In some other versions, distal end (322) may be slightly backed out proximally, such that distal end (322) does not contact fundus (F) throughout the rest of the procedure shown in FIGS. 6B-6E.

After reaching the state shown in FIG. 6B, balloon (324) may be inflated as described above; and as shown in FIG. 6C. In some cases, balloon (324) is inflated to a point where balloon (324) bears outwardly against the sidewall of the uterus (U). In any case, the inflated balloon (324) may stabilize the distal portion of shaft (320) relative to the uterus (U). Specifically, the inflated balloon (324) may prevent shaft (320) from exiting proximally from the uterus (U) via the cervix (C). Balloon (324) may thus serve as a distally-positioned anchor structure for uterine manipulator (300). The inflated balloon (324) may also provide sufficient engagement between shaft (320) and the uterus (U) to allow use of shaft (320) to reposition and reorient the uterus (U) as described herein.

With balloon (324) in the inflated state the operator may advance sleeve (330) distally along shaft (320) to the position shown in FIG. 6D. In the present example, this is performed by a human operator manually advancing sleeve (330) distally along shaft (320). In some other versions, this may be performed by a robotic operator robotically advancing sleeve (330) distally along shaft (320). As shown, sleeve (330) is advanced distally to a point where distal end (360) is firmly seated in the vaginal fornix (VF). The cervix (C) is received in interior space (354) of body (352). At this stage, the longitudinal position of sleeve (330) along shaft (320) is locked in place via locking ring (340). Specifically, the operator grasps locking ring (340) and rotates locking ring (340) about shaft (320) to firmly lock the position of sleeve (330) along shaft (320). In the present example, this is performed by a human operator, though it may be performed by a robotic operator in other versions. With the position of sleeve (330) locked in place against shaft (320), the position of uterine manipulator (300) is substantially fixed relative to the vagina (V), the cervix (C), and the uterus (U). While balloon (324) serves as a distally-positioned anchor structure for uterine manipulator (300), colpotomy cup (350) serves as a proximally-positioned anchor structure for uterine manipulator (300).

With the position of uterine manipulator (300) being fixed by the combination of balloon (324) and colpotomy cup (350), balloon (332) is inflated as shown in FIG. 6E. Balloon (332) bears outwardly against the sidewall of the vagina (V), thereby creating a fluid-tight seal against the sidewall of the vagina (V).

With uterine manipulator (300) being positioned and configured as shown in FIG. 6E, robotic arm (110) may be utilized to drive uterine manipulator (300) to various positions, to thereby re-orient and reposition the uterus (U) as desired by the clinician who is performing the rest of the medical procedure (e.g., hysterectomy). In some scenarios, the clinician who robotically controls robotic arm (110) to drive uterine manipulator (300) to position and orient the uterus (U) also uses the same robotic system to control instruments that are used to perform a surgical procedure associated with the uterus (U) (e.g., a hysterectomy).

As noted above, by allowing a surgeon to directly control the manipulation of the uterus (U) via robotic arm (110) and uterine manipulator (300), the process avoids potential confusion and inconsistency that might otherwise result in procedures where a human assistant is controlling a uterine manipulator based on commands from another human clinician. Moreover, once the uterus (U) has been manipulated to achieve the desired position and orientation, robotic arm (110) and uterine manipulator (300) may cooperate to maintain this position and orientation of the uterus (U) indefinitely. This may avoid scenarios where a human operator of a uterine manipulator (300) might inadvertently reposition or reorient the uterus (U) in the middle of a medical procedure.

As noted above, one medical procedure that may be performed using robotic arm (110) and uterine manipulator (300) is a hysterectomy. In some versions of such a procedure, one or more cutting instruments are introduced laparoscopically via the patient's abdomen to approach the cervicovaginal junction from outside the uterus (U) and vagina (V). Such instrumentation may be controlled manually or robotically. In versions where the instrumentation is controlled robotically, the same robotic system may control the instrumentation and robotic arm (110). A cutting instrument may cut the uterus (U) away at the cervicovaginal junction, generally tracing around the circular perimeter defined by distal end (360) of colpotomy cup (350).

This cutting at the cervicovaginal junction will ultimately result in separation of the uterus (U) from the vagina (V); and the end of the vagina (V) may be appropriately closed at this point. During this process, the patient's abdomen may be insufflated with pressurized gas, and the pressurized insufflation gas may eventually reach the distal region of the vagina (V). In such scenarios, balloon (332) will provide sealed occlusion that is sufficient to prevent the pressurized insufflation gas from escaping out of the patient via the vagina (V).

While robotic arm (110) and uterine manipulator (300) are described in the foregoing example as being used in a hysterectomy, robotic arm (200) and uterine manipulator (300) may be used in any other suitable fashion and may be used in any other suitable procedures.

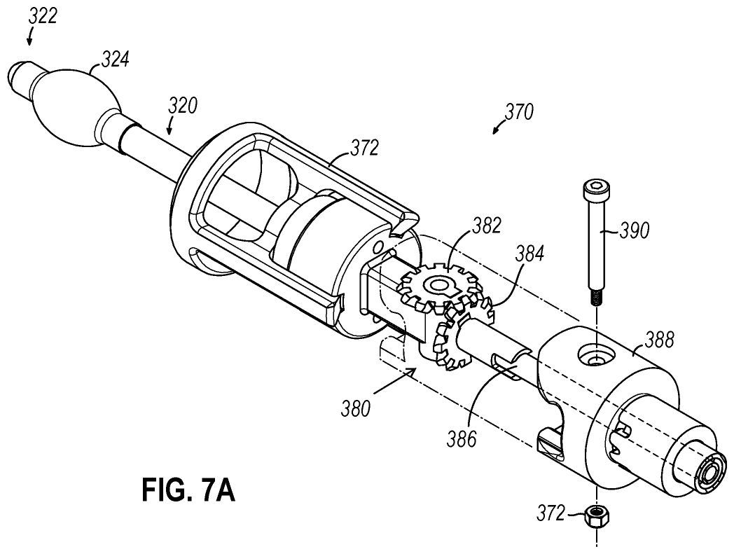

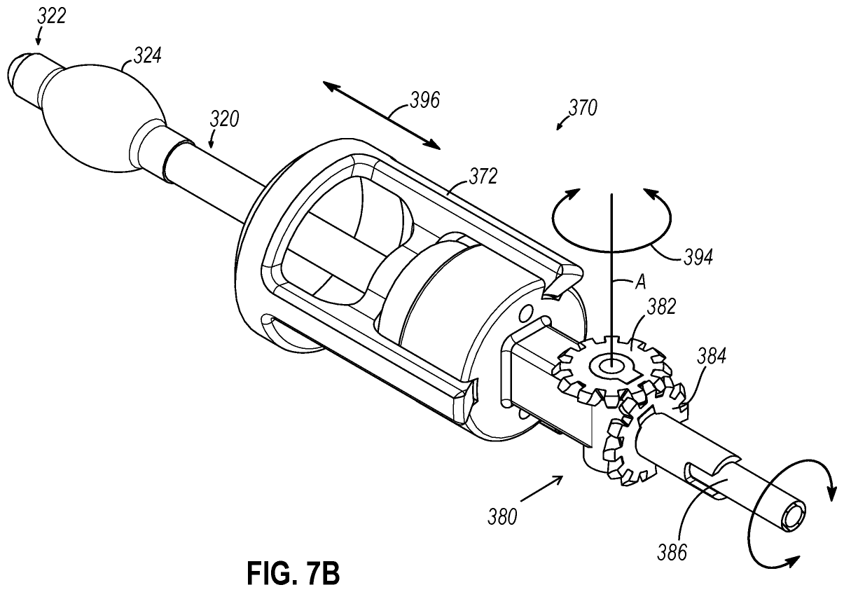

In some instances, it may be desirable to control the position and orientation of the uterus (U) without moving the proximal portion of uterine manipulator (300) relative to the patient. FIGS. 7A-7B show an alternative colpotomy cup (370) that may be readily incorporated into uterine manipulator (300) described above in replacement of colpotomy cup (350). Therefore, colpotomy cup (370) may translate into a suitable longitudinal position relative to shaft (320), as illustrated by arrow (396) in FIG. 7B, in order to suitably position colpotomy cup (370) relative to shaft (320) in accordance with the description herein.

Colpotomy cup (370) is substantially similar to colpotomy cup (370) described above, with differences elaborated below. In particular, colpotomy cup (370) includes an articulation drive assembly (380) configured to rotate body (372) of colpotomy cup (370) about articulation axis (A), as illustrated by arrow (394), relative to a proximal portion of shaft (320). Shaft (320) extends through colpotomy cup (370) such that rotation of colpotomy cup (370) about articulation axis (A) bends distal end (322) and balloon (324) of shaft (320). Therefore, articulation drive assembly (380) may be utilized to control the position and orientation of the uterus (U) via articulation of colpotomy cup (370) and distal end (322).

In the current example, articulation drive assembly (380) includes a first gear (382), a second gear (384) meshed with the first gear (382), a rotary driver (386) extending proximally within shaft (320) and attached to second gear (384), and a clevis body (388) housing gears (382, 384) via a pin (390) and nut (392). Pin (390) extends through an opening defined by first gear (382), a proximal portion of body (372), and clevis body (388).

Rotary driver (386) extends through clevis body (388) and is configured to rotate relative to clevis body (388) about its own axis. Rotary driver (386) is suitably attached to a rotating input (not shown) located at a proximal end of uterine manipular (300). Rotating input (not shown) is configured to drive rotation of rotary driver (386) about its own longitudinal axis, thereby driving rotation of second gear (384) relative to clevis body (388). Second gear (384) suitably meshes with first gear (382) such that rotation of second gear (384) via rotary driver (386) drives rotation of first gear (382) as indicated by the arrow (394). Therefore, second gear (384) may rotate first gear (382) in a first rotational direction, or an opposite, second, rotational direction. First gear (382) is suitably coupled to body (372) of colpotomy cup (370) such that rotation of first gear (382) drives rotation of body (372) about articulation axis (A) relative to clevis body (388) and the proximal portion of shaft (320), as indicated by arrow (394) shown in FIG. 7B. Therefore, an operator may utilize articulation drive assembly (380) in order to articulate distal end (322) of shaft (320) and colpotomy cup (370) relative to a proximal portion of shaft (320). Such articulation may be utilized when colpotomy cup (370) and shaft (320) are suitably attached to anatomy of a patient in order to position and orient the uterus (U) in accordance with the description herein.

### III. Examples of Features to Restrict Range of Motion of Components in Robotically Surgical System

As mentioned above, robotic arm (110) may be utilized to control uterine manipulator (300) during use in a medical procedure. For example, robotic arm (110) may be utilized to move uterine manipulator (300), while suitably anchored to uterus (U), in order to reposition and reorient the uterus (U) as described in accordance herein. Additionally, or alternatively, robotic arm (110) may be utilized to advance/retract uterine manipulator (300), in part or in its entirety, within uterus (U) in order to suitably anchor uterine manipulator (300) in accordance with the description herein. As also mentioned above, robotic arm (110) may be positioned between the legs of a patient while controlling uterine manipulator (300) in accordance with the description herein.

Movement of robotic arm (110) may be controlled by a clinician via control console (28). Control console (28) may include any suitable components as would be apparent to one skilled in the art in view of the teachings herein, such as processor(s), memory, storage, visual display units(s), controllers, input devices, point and click devices, monitoring units, mechanical drivers, generators, etc. In some instances, portions of control console (28) utilized by the clinician to control movement of robotic arm (110) are located outside the surgical theater where robotic arms (110) and the patient are located. Control console (28) may include separate modules that are in communication with each other but located at different areas/rooms/geographical locations. For example, control console (28) may include a surgeon's control console configured to be utilized by the clinician to control movement of robotic arm(s) (110); and also a designated control tower located near platform (14) for use by clinicians within the surgical theater.

In instances where robotic arm (110) effectively replaces a human clinician who would otherwise be tasked with controlling uterine manipulator (300), robotic arm (110) may not possess the spatial awareness and/or ability to receive tactile feedback (such as feeling sudden resistance to movement of uterine manipulator (300) within the patient) as compared to a human clinician manually controlling uterine manipulator (300). Therefore, robotic arm (110) may inadvertently apply an undesirable amount of force to anatomy (either internally or externally) while attempting to control uterine manipulator (300). For example, robotic arm (110) may actuate uterine manipulator (300), while anchored to the uterus (U) in accordance with the description herein, without appreciating anatomical resistance to such actuation. As another example, in instances where robotic arm (110) is located between legs of the patient, robotic arm (110) may inadvertently contact a leg of the patient while actuating uterine manipular (300).

Therefore, it may be desirable for robotic system (10) to determine an internal and/or external volume/zone of operation where uterine manipulator (300) and/or robotic arm (110) may move. Restricting movement of robotic arm (110) and uterine manipulator (300) to be confined within certain internal and/or external zones of operation during illustrative use may prevent robotic arm (110) and uterine manipulator (300) from inadvertently applying an undesirable amount of force to anatomy. Further, it may be desirable to confine movement of uterine manipulator (300) and/or robotic arm (110) within such volumes of operations such that a clinician controlling robotic arm (110) and uterine manipulator (300) may not generate control signals that inadvertently actuate manipulator (300) and/or robotic arm (110) outside such determined zones.

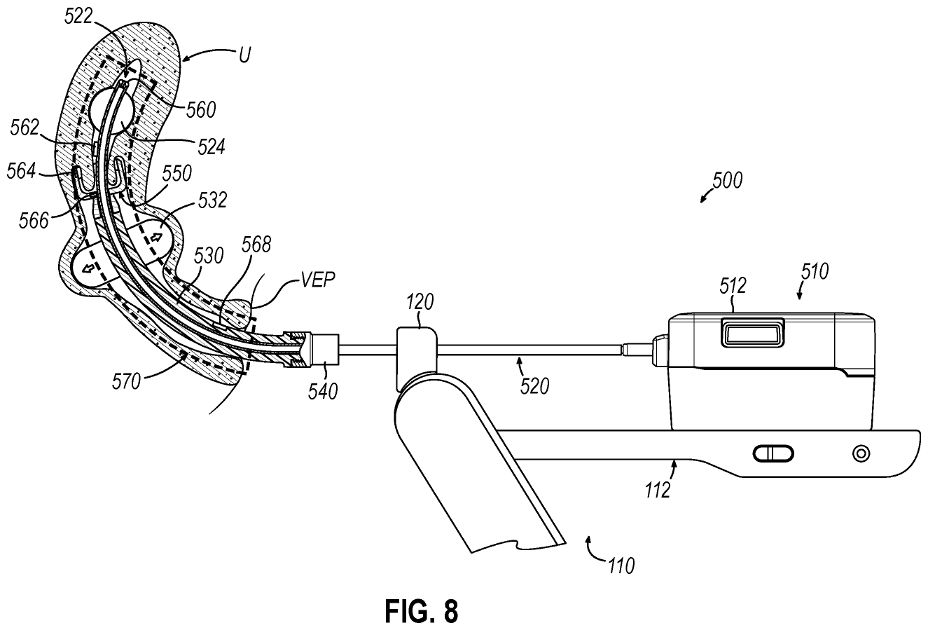

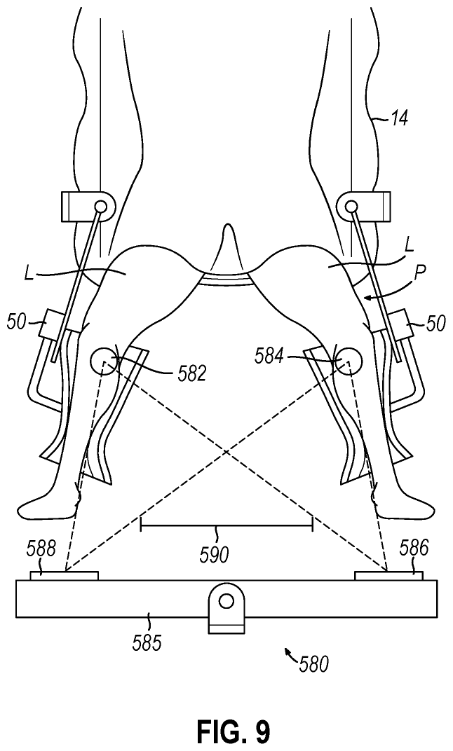

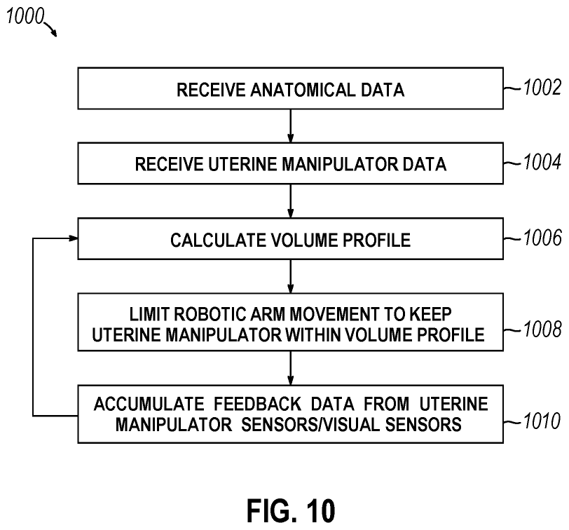

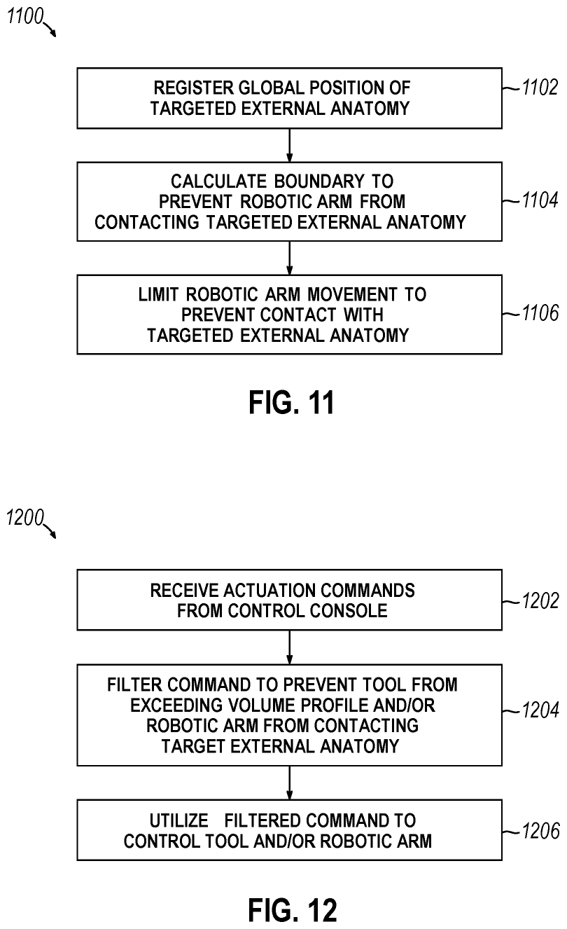

FIGS. 8 and 9 show an example of a uterine manipulator (500) and a visual tracking system (580), respectively; while FIGS. 10 and 11 show examples of zone of operation algorithms (1000, 1100) for robotic system (10) to determine and utilize internal zones of operation (570) (see FIG. 8) and external zones of operation (590) (see FIG. 9) during a surgical procedure. Further, FIG. 12 shows an example of a filtering algorithm (1200) for robotic system (10) to prevent robotic arm (110) and uterine manipulator (300) from undesirably departing from such zones of operation during a surgical procedure.

As will be described in greater detail below, uterine manipulator (500) and visual tracking system (580) may be utilized with robotic system (10) in conjunction with algorithms (1000, 1100, 1200) during a surgical procedure such that robotic arm (110) and uterine manipulator (500) operate within the boundaries of external and/or internal zones of operation. Keeping robotic arm (110) within an external zone of operation (590) (see FIG. 9) may prevent robotic arm (110) from inadvertently contacting either patient or other structures (such as stirrups (50) shown in FIG. 9) during illustrative use in accordance with the description herein. Further, keeping uterine manipulator (500) within an internal zone of operation (570) (see FIG. 8) may prevent uterine manipulator (500) from imparting undesirable excessive forces on anatomical structures during use in accordance with the description herein.

Turning to FIG. 8, uterine manipulator (500) may be substantially similar to uterine manipulator (300) described above, with differences elaborated below. Uterine manipular (500) thus includes a tool driver interface (510), a base (512), a shaft (520), a distal end (522), a balloon (524), a sleeve (530), a balloon (532), a locking ring (540), and a colpotomy cup (550); which may be substantially similar to tool driver interface (310), base (312), shaft (320), distal end (322), balloon (324), sleeve (330), balloon (332), locking ring (340), and colpotomy cup (350) described above, with differences elaborated below.

Base (512) is operatively coupled to tool driver (112) while a proximal portion of shaft (520) extends though cannula (120). In some examples, tool driver (112) may acuate base (512) in the proximal and distal directions such that shaft (520) is slidably associated with cannula (120). In some examples, base (512) may be longitudinally fixed on tool driver (112) such that shaft (520) does not slide within cannula (120). Uterine manipulator (500) is suitably attached to robotic arm (110) such that robot arm (110) may actuate uterine manipulator (500) in order to suitably position and orient uterus (U) in accordance with the description herein.

Uterine manipulator (500), in the current example, includes a distal shaft force sensor (560), a lateral shaft force sensor (562), a sidewall colpotomy cup force sensor (564), a floor colpotomy cup force sensor (566), and a lateral sleeve force sensor (568). Each force sensor (560, 562, 564, 566, 568) is configured to measure forces that anatomical structures impart on the component to which force sensor (560, 562, 564, 566, 568) is attached. Such forces imparted on sensors (560, 562, 564, 566, 568) by anatomical structures may be reactionary forces in response to robotic arm (110) actuating uterine manipulator (500) within the uterus (U). Therefore, force sensors (560, 562, 564, 566, 568) are configured to effectively measure the forces that uterine manipulator (500) imparts on anatomical structures in response to robotic arm (110) actuating manipulator (500).

Distal shaft force sensor (560) may be configured to measure an axial compressive force generated when distal end (522) of shaft engages fundus (F) of the uterus (U). Lateral shaft force sensor (562) may be configured to measure a lateral force imparted on shaft (520) in response to uterine manipulator (500) repositioning uterus (U) while uterine manipulator (500) is anchored to uterus (U) in accordance with the description herein. Sidewall colpotomy cup force sensor (564) may be configured to measure forces acting on the sidewall of colpotomy cup (550) due to engagement with vaginal fornix (VF) and/or cervix (C). Floor colpotomy cup force sensor (566) may be configured to measure compressive forces generated by cervix (C) engaging the floor of colpotomy cup (550). Lateral sleeve force sensor (568) may be configured to measure a lateral force imparted on sleeve (530) in response to uterine manipulator (500) repositioning uterus (U) while uterine manipulator (500) is anchored to uterus (U) in accordance with the description herein. Force sensors (560, 562, 564, 566, 568) may be configured to measure any other suitable forces as would be apparent to one skilled in the art in view of the teachings herein.

Each force sensor (560, 562, 564, 566, 568) is in communication with corresponding components of robotic system (10), such as a designated control tower of control console (28). Communication between force sensor (560, 562, 564, 566, 568) and corresponding components of robotic system (10) may be established through any suitable means as would be apparent to one skilled in the art in view of the teachings herein. For example, electrical traces may extend from each sensor (560, 562, 564, 566, 568) into base (512) such that base (512) may establish communication with robotic system (10) via tool driver (112) of robotic arm (110) when suitably coupled (as shown in FIG. 8). As another example, sensors (560, 562, 564, 566, 568) may utilize suitable wireless technology to establish communication with suitable components of robotic system (10).

While all force sensors (560, 562, 564, 566, 568) are present in the current example, uterine manipulator (500) may include any suitable combination of force sensors (560, 562, 564, 566, 568) as would be apparent to one skilled in the art in view of the teachings herein, such that one or more of force sensors (560, 562, 564, 566, 568) may be omitted; and such that one or more other force sensors may be included. Additionally, an array of force sensors may be attached to their respective component, rather than the single force sensor shown in the current example. Alternatively, in some examples, force sensors (560, 562, 564, 566, 568) may not be required, such that force sensors (560, 562, 564, 566, 568) are omitted.

As will be described in greater detail below, robotic system (10) may utilize data provided by force sensors (560, 562, 564, 566, 568) in order to generate and/or recalibrate an internal volume of operation (570). Additionally, or alternatively, as will also be described in greater detail below, anatomical data of the patient and/or tool size(s) data of uterine manipulator (500) may be utilized by robotic system (10) in order to generate internal volume of operation (570).

FIG. 10 shows an example of an internal zone of operation algorithm (1000) that may be utilized by robotic system (10) in order to generate and utilize internal volume of operation (570). Suitable components of robotic system (10), such as a designated control tower of control console (28), may receive anatomical data (1002) related to the patient. Anatomical data (1002) may be uploaded manually by a clinician, may be uploaded automatically from a patient specific file, or may be uploaded automatically from visual data obtained during or prior to the surgical procedure. Any suitable anatomical data (1002) may be utilized as would be apparent to one skilled in the art in view of the teachings herein. For example, the depth of the uterus (U) determined during a sounding process may be utilized, the size the cervix (C), the dimensions of the vaginal canal may be utilized, etc.

Additionally, suitable components of robotic system (10) receive uterine manipulator data (1004) related to the specific uterine manipulator (500) attached to robotic arm (110) for the specific procedure. Uterine manipulator data (1004) may include dimensions of various components of uterine manipulator (500). For example, the dimensions of distal tip (522), shaft (520), and colpotomy cup (550) may be included in uterine manipulator data (1004). Uterine manipulator data (1004) may include other suitable data regarding the specific uterine manipulator (500) being used as would be apparent to one skilled in the art in view of the teachings herein. For example, uterine manipulator data (1004) may include the manufacturer and model of uterine manipulator (500), the type of material used for various components of uterine manipulator (500), whether or not uterine manipulator (500) is articulatable, etc.

Manipulator data (1004) may be uploaded manually to suitable components of robotic system (10) by a clinician. For example, a clinician may upload specific characteristics of manipulator data (1004) as prompted by robotic system (10) utilizing algorithm (1000). As another example, a clinician may enter a model number, thereby allowing robotic system (10) to upload manipulator data (1004) associated with such a model number. Additionally or alternately, uterine manipulator (500) may be uploaded automatically once manipulator (500) is coupled to robotic arm (110). Such data may be transmitted via a mechanical switch, an electrical switch, Bluetooth, etc. As one example, specific manipulators (500) with certain characteristics may be configured to activate a specific switch (either mechanical or electrical) located on robotic arm (110). Activation of such a specific switch may indicate to robotic arm (110) and system (10) that a uterine manipulator (500) with specific characteristics has been attached to robotic arm (110). Therefore, robotic system (10) may then infer uterine manipulator data (1004) based on which switch is activated. As another example, uterine manipulator may contain suitable data (1004) stored on a memory or storage unit located within base (110) of manipulator (500). Such data (1004) may be uploaded to robotic system (10) once manipulator (500) is attached to robotic arm (110) via an electrical or wireless connection.

Next, suitable components of robotic system (10) may utilize data (1002, 1004) provided in order to calculate an initial volume profile (1006) associated with an internal zone of operation (570). In some examples, robotic system (10) may utilize the calculated volume profile (1006) to define the internal zone of operation (570) after uterine manipulator (500) is suitably anchored to uterus (U) in accordance with the description herein. Therefore, after uterine manipulator (500) is anchored to uterus (U), robotic system (10) may superimpose the calculated volume profile (1006) over the determined initial anchored location of uterine manipulator (500) in order to define the internal zone of operation (570) as shown in FIG. 8.

With the volume profile (1006) calculated and the internal zone of operation (570) established, the clinician in control of robotic arm (110) may instruct robotic arm (110) to actuate uterine manipulator (500) to thereby position and orient uterus (U) in accordance with the description herein. Robotic system (10) may automatically restrict robotic arm movement (1008), despite contrary user input that would otherwise drive uterine manipulator (500) outside the internal zone of operation (570), to keep uterine manipulator (500) (or selective portions of uterine manipulator (500), such as shaft (520)) within the internal zone of operation (570). Robotic system (10) may utilize filtering algorithm (1200) shown in FIG. 12 to limit robotic arm (110) movement.

Thus, if the clinician in control of robotic arm (110) provides instructions that direct robotic arm (110) to actuate selective portions of manipulator (500) (such as shaft (520) and/or distal tip (522)) out of the internal zone of operation (570), robotic system (10) will prevent such movement from occurring. As mentioned above, keeping selective portions (such as distal tip (522), colpotomy cup (550), and/or selective portions of shaft (520)) of uterine manipulator (500) within the confines of the internal zone of operation (570) prevents uterine manipulator (500) from imparting undesirable excessive forces on anatomical structures during use in accordance with the description herein.

Additionally, during use, robotic system (10) may accumulate feedback data (1010) from force sensors (560, 562, 564, 566, 568), suitable visual sensors, or any other suitable sensors as would be apparent to one skilled in the art in view of the teachings herein. In addition to being obtained through one or more sensors, or in lieu of being obtained through one or more sensors, the feedback data may be based at least in part on instrument kinematics gathered based on movements of different components of robotic arm (110) during operation. In any case, robotic system (10) may utilize the accumulated feedback data (1010) in order to modify and/or recalculate the volume profile (1006) used to determine internal zone of operation (570). Robotic system (10) may then utilize the modified and/or recalculated newly calculated volume profile (1006) to update the internal zone of operation (570) which robotic arm (110) is limited.

For example, if robotic arm (110) is instructed to move uterine manipulator (500) into a position that is well within the current internal zone of operation (570), but accumulated feedback data (1010) suggests manipulator (500) is imparting an undesirable amount of force onto anatomical structures, robotic system (10) may modify the volume profile (1006) used to determine the internal zone of operation (570) to further restrict such movement. As another example, if robotic arm (110) is instructed to move uterine manipulator (500) out of a position within the current internal zone of operation (570), but accumulated feedback data (1010) suggests manipulator (500) is not imparting an undesirable amount of force onto anatomical structures, robotic system (10) may modify the volume profile (1006) used to determine the internal zone of operation (570) to further allow such movement. Therefore, algorithm (1000) is configured to adjust the volume profile (1006) of the internal zone of operation (570) in real time based on updated data accumulated during illustrative use in accordance with the description herein.

In some instances, a laparoscopic camera may visually track the position of colpotomy cup (550) and limit the movement of colpotomy cup (550) within internal zone of operation (570). Visual tracking of colpotomy cup (550) may be achieved by visually identifying the bulge created by colpotomy cup (550) engaging the vaginal fornix (VF), or by colpotomy cup (550) emitting light that may be viewed by the laparoscopic camera through the uterus (U). Other suitable means of visually tracking colpotomy cup (550) may be utilized as would be apparent to one skilled in the art in view of the teachings herein.

While in the current example, anatomical data (1002), uterine manipulator data (1004), and feedback data (1010) is utilized in order to calculate internal volume profile (1006); any suitable single source of data (1002, 1004, 1010) or suitable combination of data (1002, 1004, 1010) may be utilized to calculate internal volume profile (1006) as would be apparent to one skilled in the art in view of the teachings herein. For example, uterine manipulator data (1004) and feedback data (1010) may be utilized to determine internal volume profile (1006). As another example, just uterine manipular data (1004) and anatomical data (1002) may be utilized in order to determine internal volume profile (1006). In some instances, internal volume profile (570) may be established uniformly such that volume profile (570) may be safe for a substantially wide range of anatomical sizes.

As mentioned above, visual tracking system (580) (see FIG. 9) and algorithm (1100) (see FIG. 11) may be utilized to determine and utilize external zone of operation (590) (see FIG. 9) to inhibit robotic arm (110) from inadvertently contacting either patient or other structures (such as stirrups (50) shown in FIG. 9) during illustrative use in accordance with the description herein. Turning to FIG. 9, visual tracking system (580) includes a base structure (585) having a first visual tracker (586) and a second visual tracker (588) located on opposite end of base structure (585) yet facing in the same general direction. By way of example only, each visual tracker (586, 588) may comprise one or more cameras or other forms of optical sensor(s).

Base structure (585) may be located on a stand configured to be moved around in the surgical theater. Therefore, base structure (585) may be moved to various desirable locations within the surgical theater. Base structure (585) and/or visual trackers (586, 588) may be in communication with suitable components of robotic system (10) such that base structure (585) and/or visual trackers (586, 588) may communicate its global reference position to robotic system (10) within the surgical theater. In other words, robotic system (10) is configured to track the position of base (585) and/or visual tracker (586, 588) within the surgical theater.

Visual trackers (586, 588) are configured to visually identify suitable objects within the surgical theater and determine a distance between visual trackers (586, 588) and the visually identified objects. Visual trackers (586, 588) are further configured to communicate the distance between visual trackers (586, 588) and visually identified objects to robotic system (10). Since robotic system (10) knows the global position/location of visual trackers (586, 588) within the surgical theater; robotic system (10) may utilize the global position of visual trackers (586, 588) and the relative position of visually identified objects relative to visual trackers (586, 588) in order to calculate to global position of visually identified objects within the surgical theater.

As shown in FIG. 9, a first fiducial (582) and a second fiducial (584) may be placed on the legs (L) of patient (P) (e.g., on or near each knee of the patient (P)), while visual trackers (586, 588) may be positioned adjacent to patient (P) such that visual trackers (586, 588) may determine the distance between trackers (586, 588) and each fiducial (582, 584). In other words, fiducials (582, 584) and visual trackers (586, 588) are arranged such that fiducials (582, 584) are within the fields of view of visual trackers (586, 588). Therefore, it should be understood fiducials (582, 584) are configured to be visually identified and tracked by visual trackers (586, 588).

Robotic system (10) may utilize the location between visual trackers (585, 588) and each fiducial (582, 584) in order to determine the global position of each fiducial (582, 584) within the surgical theater in accordance with the description herein. With the global position of each fiducial (582, 584) known, robotic system (10) may also calculate the lateral distance between fiducials (582, 584). Fiducials (582, 584) may be placed at any suitable location relative to patient (P) as would be apparent to one skilled in the art in view of the teachings herein. For example, fiducials (582, 584) may be placed on stirrups (50).

Turning to FIG. 11, robotic system (10) may utilize algorithm (1100) to generally register the global position of targeted external anatomy (1102) by determining the global location of fiducials (582, 584) within surgical theater. Fiducials (582, 584) are located at suitable locations on or near legs (L) of patient (P) such that the location of fiducials (582, 584) generally represent the location of legs (L) (i.e., the targeted external anatomy in this example).

In some instances, fiducials (582, 584) and visual tracking system (580) are temporarily used prior to the surgical procedure in order to register the global position of the targeted external anatomy (1102), and then removed. Fiducials (582, 584) may include any suitable structures as would be apparent to one skilled in the art in view of the teachings herein, including but not limited to spheres or other objects having known shapes. In some instances, fiducials (582, 584) may be permanent features of suitable structures, such as stirrups (50); such that only visual tracking system (580) is removed after registering the global position of the targeted external anatomy (1102).

In still other variations, fiducials (582, 584) are not used at all to register the global position of the targeted external anatomy (1102). For instance, one or more visual trackers (586, 588) may obtain one or more images of one or more external anatomical structures of the patient; and a processor (e.g., within control console (28)) may utilize image recognition or other image processing from image data captured by one or more visual trackers (586, 588) to register the global position of the targeted external anatomy (1102). It should therefore be understood that fiducials (582, 584) are not necessarily required in all variations. It should also be understood that anatomical data received in advance of the procedure may be utilized in registering the global position of the targeted external anatomy (1102).

Next, with the location of targeted external anatomy registered (1102), robotic system (10) may then utilize the registered position of external anatomy to calculate the boundary (1104) to prevent robotic arm (110) from contacting target external anatomy. In the current example shown in FIG. 9, the calculated boundary (1104) coincides with the external zone of operation (590). External zone of operation (590) is a lateral space between legs (L) which robotic arm (110) may actuate between without undesirably contacting legs (L). Therefore, during the surgical procedure, the clinician in control of robotic arm (110) may instruct robotic arm (110) to actuate in accordance with the description herein. Robotic system (10) may then limit robotic arm movement (1006) to prevent robotic arm (110) from departing from external zone of operation (590). Robotic system (10) may utilize filtering algorithm (1200) shown in FIG. 12 to limit robotic arm (110) movement.

It should therefore be understood that fiducial (582, 584) and visual tracking system (580) (and/or other equipment and/or other techniques) may provide a global location of legs (L) of patient (P) for robotic system (10) to utilize. Further, robotic system (10) may utilize the registered global position of legs (L) to calculate a boundary (1104) for robotic arm (110) to move between during illustrative use in accordance with the description herein. Robotic system (10) may further limit movement of such a robotic arm (110) to prevent robotic arm (110) from leaving external zone of operation (590) coinciding with the calculated lateral boundary (1104).

As mentioned above, both algorithms (1000, 1100) may utilize filtering algorithm (1200) that inhibits robotic arm (110) to actuate out of external zone of operation (590), or robotic arm (110) to actuate select portions of uterine manipulator (500) outside internal zone of operation (570). Turning to FIG. 12, filtering algorithm (1200) includes receiving actuation commands (1202) from control console (28). As mentioned above, actuation commands (1202) may be configured to move robotic arm (110) during illustrative use in accordance with the description herein. Next, algorithm (1220) filters (1204) such actuation commands (1202) and utilizes the filtered commands (1206) to control uterine manipulator (500) and/or robotic arm (110).

In some instances, if the received actuation commands (1202) instructs robotic arm (110) and/or uterine manipulator (500) to depart from the current zones of operation (570, 590), the filtering process (1204) may either negate such commands entirely such that robotic arm (110) does not move at all. In other instances, the filtering process (1204) may modify such commands such that robotic arm (110) actuates toward the boundary of zones of operation (570, 590) but does not depart/exit from such boundary.

IV. Example of Robotically Controlled Surgical Instrument with Docking Alignment Features

As mentioned above, in some instances, uterine manipulator (300, 500) is suitably anchored to the uterus (U) of a patient via a second clinician manually controlling uterine manipulator (300, 500). Once the second clinician suitably anchors uterine manipulator (300, 500) in accordance with the description herein, uterine manipulator (300, 500) may be attached to robotic arm (110) (i.e., docked to robotic arm (110)). Docking uterine manipulator (300, 500) to robotic arm (110) may allow robotic arm (110) to stabilize uterine manipulator (300, 500) such that the second clinician does not need to constantly support uterine manipulator (300, 500) during the procedure. Additionally, docking uterine manipulator (300, 500) allows the first clinician to control the position and orientation of the uterus of the patient via movement of robotic arm (110).

However, when docking an already anchored uterine manipulator (300, 500) to robotic arm (110), if the interfacing components of manipulator (300, 500) and robotic arm (110) (e.g., interfacing surfaces of base (312, 512) and tool driver (112)) are not suitably aligned, initial engagement between interfacing components of manipulator (300, 500) and robotic arm (110) may cause robotic arm (110) to undesirably drive movement of an anchored uterine manipulator (300, 500). Such undesirable movement of an anchored uterine manipulator (300, 500) may undesirably affect the anchoring of uterine manipulator (300, 500). Therefore, it may be desirable to have a uterine manipulator with docking alignment features that promotes proper alignment between interfacing components of uterine manipular and robotic arm (110).

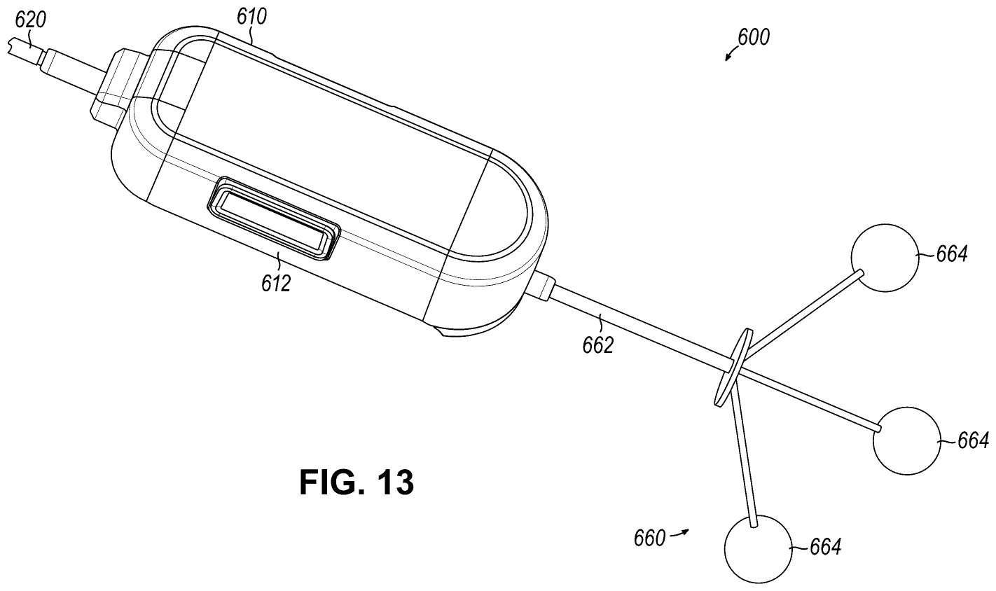

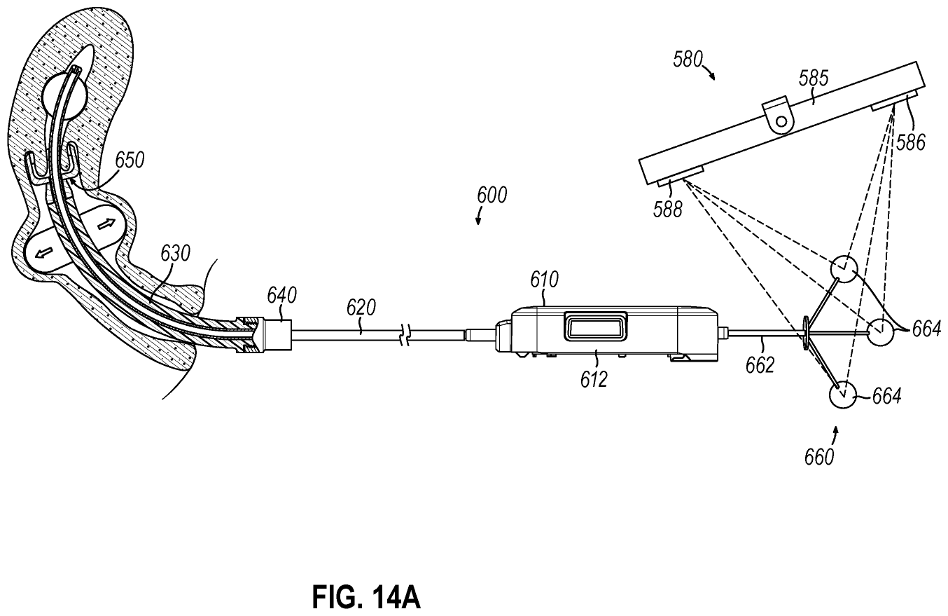

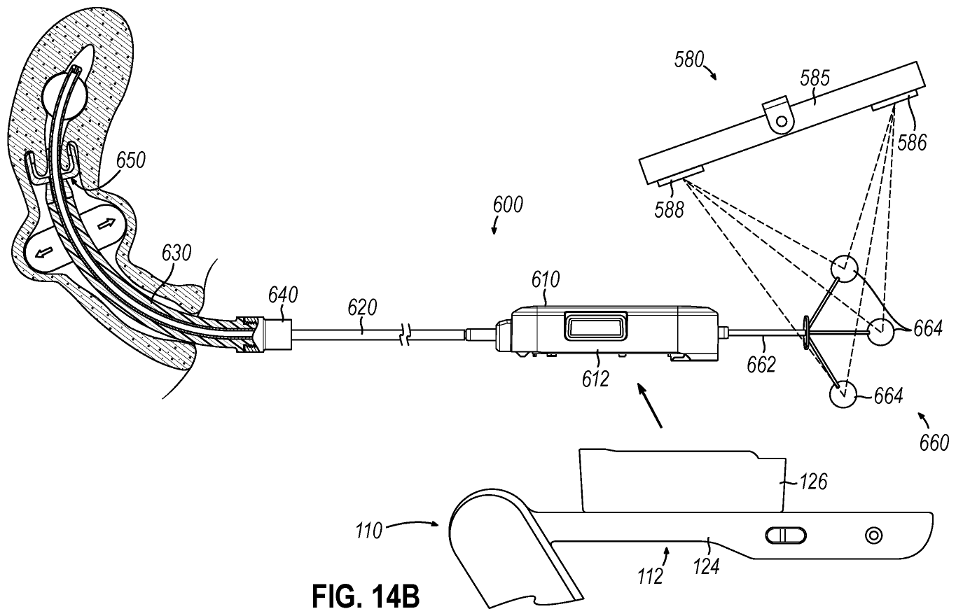

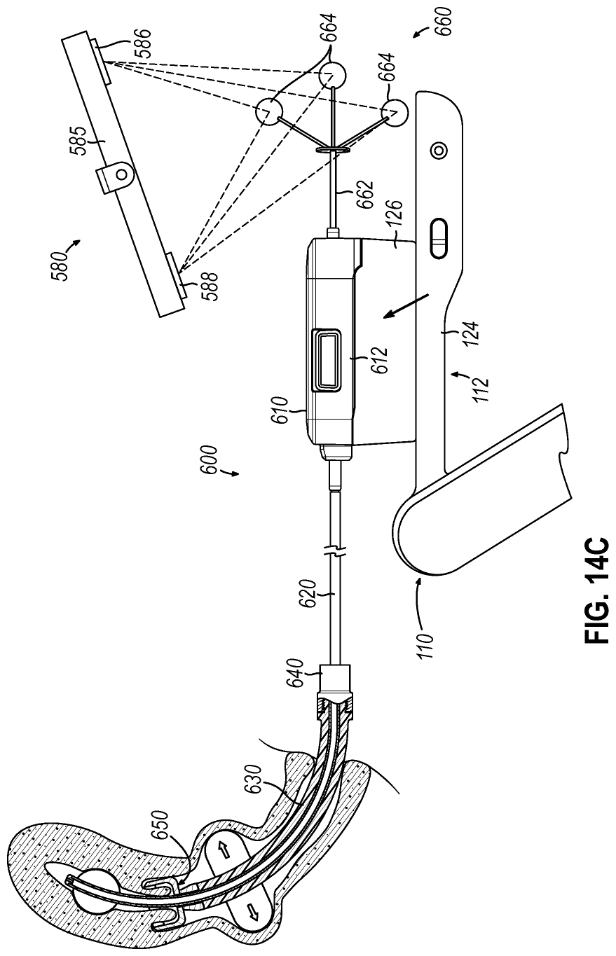

FIGS. 13-14C show an example uterine manipulator (600), which may be substantially similar to uterine manipulator (300, 500) described above, with differences elaborated below. Uterine manipulator (600) thus includes a tool driver interface (610), a shaft (620), a sleeve (630), a locking ring (640), and a colpotomy cup (650); which may be substantially similar to tool driver interface (310), shaft (320), sleeve (330), locking ring (340), and colpotomy cup (350) described above, with differences elaborated below.

Uterine manipulator (600) includes a docking alignment feature, in the form of a spatial tracking structures (660). Spatial tracking structure (660) includes a proximally extending rigid body (662) and a plurality of spatially fixed fiducials (664). Proximally extending rigid body (662) is fixed relative to base (610); while spatially fixed fiducials (664) are fixed relative to proximally extending rigid body (662). Fiducial (664) are configured to be detected by visual tracking system (580) described above in accordance with the description herein. Spatially fixed fiducials (664) are aligned with each other such that once visual tracking system (580) determines the position of each fiducial (664), robotic system (10) may determine the spatial position and orientation of base (610). Therefore, by utilizing spatially fixed fiducial (664) and visual tracking system (580), robotic system (10) may calculate the global position and orientation of the interfacing surface (612) of base (610) that is configured to couple with an interfacing surface of carriage (126) of tool driver (112). Therefore, as will be described in greater detail below, robotic system (10) may instruct robotic arm (110) to suitably align and mate with interfacing surface (612) of base (610). Further, as will be described in greater detail below, robotic system (10) may determine if the docking location between robotic arm (110) and interfacing surface (612) will allow for all the permitted motion needed to suitable perform surgical procedure.

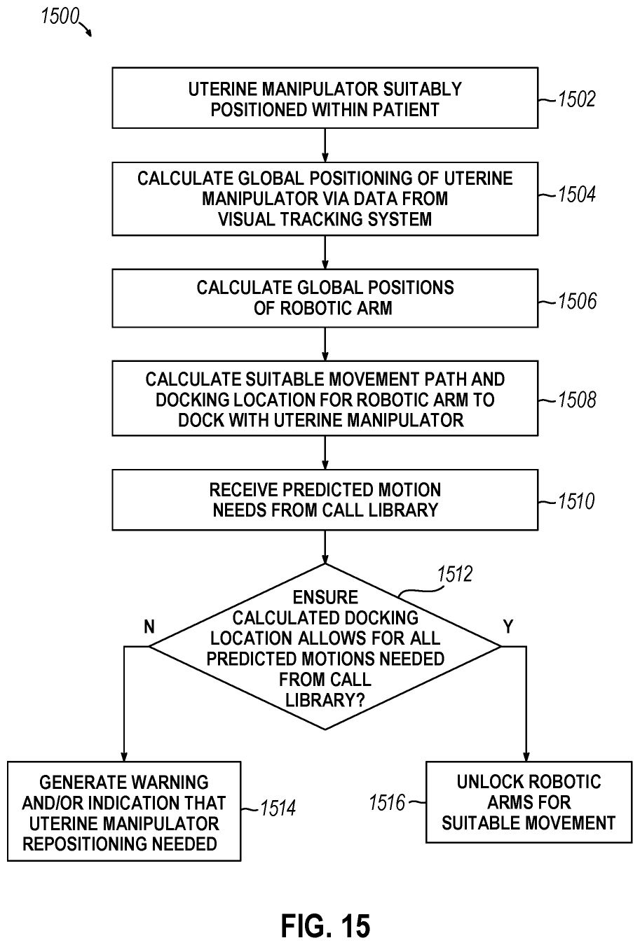

FIGS. 14A-14C show an example of use of spatial tracking structure (660) and visual tracking system (580) to suitably couple robotic arm (110) with interfacing surface (612) of base (610); while FIG. 15 shows an illustrative docking algorithm (1500) that robotic system (10) may utilize in order to dock robotic arm (110) with base (610).

First, as shown in FIG. 14A and FIG. 15, uterine manipulator (600) may be suitably positioned and anchored within patient (1502). Such positioning and anchoring is performed by a clinician. With uterine manipulator (600) suitably positioned and anchored within patient (1502), visual tracking system (580) may be utilized with robotic system (10) in order to calculate the global positioning (1504) of uterine manipulator (600). Next, robotic system (10) may calculate the global position (1506) of robotic arm (110); and as shown FIG. 15, robotic system (10) may then calculate the movement path and docking location (1508) for robotic arm (110) to dock with manipulator (600).

In order to determine whether such a docking location (1508) is suitable, robotic system (10) may receive the predicted motion needs of robotic arm (110) from a call library (1510) and ensure that the calculated docking location allows for all predicted motion needs from call library (1512). If all the predicted motion needs from call library cannot be made, robotic system (10) generates a warning and/or indication that uterine manipulator should be repositioned (1514) and the algorithm (1500) will start over. If the predicted motion needs from call library can be accomplished, robotic system will unlock robotic arm (110) for suitable movement (1516) to couple driver (112) with base (610), as shown between FIGS. 14B-14C.

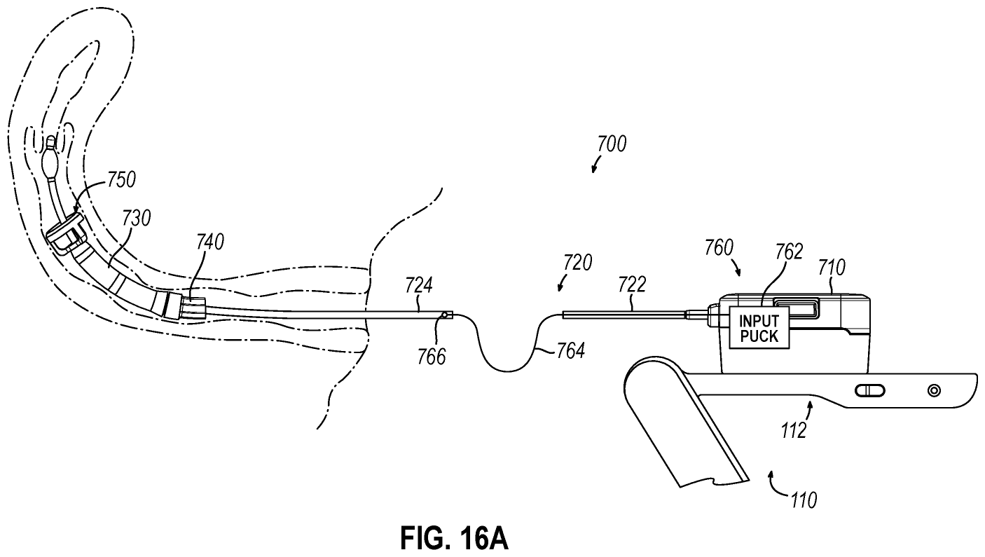

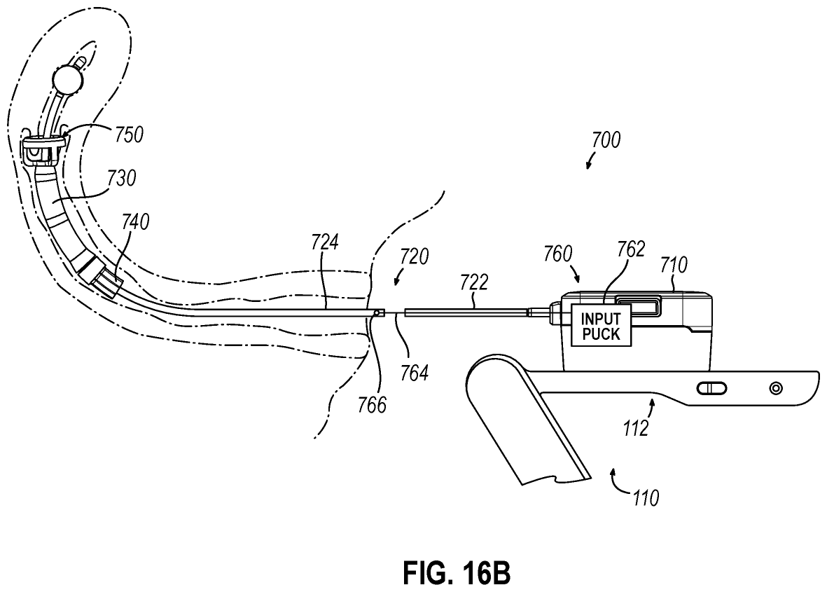

FIGS. 16A-16B show another uterine manipulator (700) with a docking alignment feature in the form a mechanical alignment and coupling assembly (760). Uterine manipulator (700) may be substantially similar to uterine manipulator (300, 500) described above, with differences elaborated below. Uterine manipulator (700) thus includes a tool driver interface (710), a shaft (720), a sleeve (730), a locking ring (740), and a colpotomy cup (750); which may be substantially similar to tool driver interface (310), shaft (320), sleeve (330), locking ring (340), and colpotomy cup (350) described above, with differences elaborated below.

Shaft (720) in the current example includes a proximal shaft (722) fixed to base (710) and a distal shaft (724) associated with sleeve (730), lock ring (740), and colpotomy cup (750). Mechanical alignment and coupling assembly (760) includes an input spool/puck (762) may wind up and feed a cable (764). Cable (764) extending through proximal shaft (722) and has an anchoring point (766) fixed to distal shaft (724). Winding cable (764) up around input puck (762) allows distal shaft (724) and proximal shaft (722) to come together. After distal shaft (724) is suitably anchored to the uterus (U), input puck (762) may wind up cable (764). Joint (118) and links (116) of robotic arm (110) may easily rotate about joints (718) as cable (764) is wound up, such that cable tension causes joints (716) to move until shafts (722, 724) are in suitable alignment for coupling.

### V. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A robotic surgical system, comprising: (a) a control console configured to generate a set of actuation instructions; (b) a robotic arm in communication with the control console, the robotic arm being configured to receive the set of actuation instructions generated by the control console, the robotic arm being configured to actuate based on the set of actuation instructions; and (c) an instrument configured to selectively attach to the robotic arm such that the robotic arm is configured to actuate the instrument based on the set of actuation instructions, the control console being configured to calculate an external zone of operation for the robotic arm based on patient tracking data, the patient tracking data indicating a position of at least one external anatomical structure of the patient, the control console being configured to calculate an instrument zone of operation for at least a portion of the instrument based on at least one of the following: (i) anatomical data received by the control console, or (ii) instrument data received by the control console.

### Example 2

The robotic surgical system of Example 1, the instrument comprising a uterine manipulator, the uterine manipulator comprising a colpotomy cup.

### Example 3

The robotic surgical system of Example 2, the instrument data comprising a size of the colpotomy cup.

### Example 4

The robotic surgical system of any of Examples 2 through 3, the uterine manipulator comprising an elongated shaft having a distal tip, the instrument data comprising a size of the distal tip of the elongated shaft.

### Example 5

The robotic system of any of Examples 2 through 4, the anatomical data comprising a size of a vaginal canal.

### Example 6

The robotic system of Example 5, the control console being configured to receive the anatomical data manually.

### Example 7

The robotic system of any of Examples 1 through 6, the control console being configured to update the instrument zone of operation based on feedback data.

### Example 8

The robotic system of Example 7, the instrument comprising at least one force sensor configured to generate the feedback data.

### Example 9

The robotic system of any of Examples 1 through 8, the instrument comprising a memory configured to upload the instrument data to the control console wirelessly.

### Example 10

The robotic system of any of Examples 1 through 9, the instrument comprising a switch configured to interact with the robotic arm in order to generate the instrument data.

### Example 11

The robotic system of any of Examples 1 through 10, the control console comprising a control tower and a surgeon control console.

### Example 12

The robotic system of any of Examples 1 through 11, further comprising a visual tracking system in communication with the control console, the visual tracking system being configured to track the position of the at least one external anatomical structure of the patient.

### Example 13

The robotic system of Example 12, the visual tracking system being configured to: (i) spatially locate at least two fiducial bodies the at least one external anatomical structure of the patient and thereby track the position of the at least one external anatomical structure of the patient, and (ii) communicate fiducial location data to the control console to thereby provide at least a portion of the patient tracking data.

### Example 14

The robotic system of any of Examples 1 through 13, the robotic arm comprising a tool driver configured to longitudinally actuate the instrument.

### Example 15

The robotic system of any of Examples 1 through 14, the instrument comprising a shaft, the shaft comprising a proximal linear portion and a distal curved portion.

### Example 16

The robotic system of any of Examples 1 through 15, the instrument comprising a base configured to selectively attach to the robotic arm.

### Example 17

A method of defining a zone of operation and limiting movement of at least a portion of an instrument within the zone of operation while the instrument is attached to a robotic arm, the method comprising: receiving a set of data associated with either (i) at least one dimension of anatomy which the instrument is to be inserted into, or (ii) at least one dimension of the instrument; calculating the zone of operation utilizing the set of data; receiving instructions which instruct the robotic arm to actuate the portion of the instrument out of the zone of operation; and filtering the received instructions such that the portion of the instrument remains within the zone of operation.

### Example 18

The method of Example 17, filtering the received instructions comprising ignoring the received instructions such that the instrument does not acuate in response to the received instructions.

### Example 19

The method of any of Examples 17 through 18, filtering the received instructions comprising modifying the received instructions such that the instrument is actuated toward a boundary of the zone of operation.

### Example 20

The method of any of Examples 17 through 19, the instrument comprising a uterine manipulator, calculating the zone of operation comprising utilizing anatomical data regarding a uterus.

### Example 21

A method of defining a zone of operation and limiting movement of a robotic arm within the zone of operation while the robotic arm is operatively coupled with an instrument, the method comprising: receiving a set of data from a visual tracking system, the set of data being associated with at least two fiducials associated with external anatomy of a patient; calculating the zone of operation utilizing the set of data; receiving instructions that instruct the robotic arm to actuate out of the zone of operation; and filtering the received instructions such that the robotic arm remains within the zone of operation.

### Example 22

A robotic surgical system, comprising: (a) a control console configured to generate a set of actuation instructions; (b) a robotic arm in communication with the control console, the robotic arm being configured to receive the set of actuation instructions generated by the control console, the robotic arm being configured to actuate based on the set of actuation instructions; (c) an instrument configured to selectively attach to the robotic arm such that the robotic arm is configured to actuate the instrument based on the set of actuation instructions; and (d) a visual tracking system in communication with the control console, the visual tracking system being configured to spatially locate at least two fiducial bodies and communicate fiducial location data to the control console, the control console being configured to calculate an external zone of operation for the robotic arm based on the fiducial location data received from the visual tracking system, the control console being configured to calculate an instrument zone of operation for at least a portion of the instrument based on at least one of the following: (i) anatomical data received by the control console, or (ii) instrument data received by the control console.

### Example 23

The robotic system of Example 22, the visual tracking system comprising a mobile unit.

### Example 24

A method of defining a zone of operation and limiting movement of a robotic arm within the zone of operation while the robotic arm is operatively coupled with an instrument, the method comprising: receiving a set of data from a visual tracking system, the set of data being associated with at least two fiducials associated with external anatomy of a patient; calculating the zone of operation utilizing the set of data; receiving instructions that instruct the robotic arm to actuate out of the zone of operation; and filtering the received instructions such that the robotic arm remains within the zone of operation.

### VI. Miscellaneous

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of uterine manipulator instruments, it should be understood that various teachings herein may be readily applied to a variety of other types of devices. By way of example only, the various teachings herein may be readily applied to other types of surgical instruments including tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A robotic surgical system, comprising:
(a) a control console configured to generate a set of actuation instructions;
(b) a robotic arm in communication with the control console, the robotic arm being configured to receive the set of actuation instructions generated by the control console, the robotic arm being configured to actuate based on the set of actuation instructions; and
(c) an instrument configured to selectively attach to the robotic arm such that the robotic arm is configured to actuate the instrument based on the set of actuation instructions
the control console being configured to calculate an external zone of operation for the robotic arm based on patient tracking data, the patient tracking data indicating a position of at least one external anatomical structure of the patient,
the control console being configured to calculate an instrument zone of operation for at least a portion of the instrument based on at least one of the following:
(i) anatomical data received by the control console, or
(ii) instrument data received by the control console.

2. The robotic surgical system of claim 1, the instrument comprising a uterine manipulator, the uterine manipulator comprising a colpotomy cup.

3. The robotic surgical system of claim 2, the instrument data comprising a size of the colpotomy cup.

4. The robotic surgical system of claim 2, the uterine manipulator comprising an elongated shaft having a distal tip, the instrument data comprising a size of the distal tip of the elongated shaft.

5. The robotic system of claim 2, the anatomical data comprising a size of a vaginal canal.

6. The robotic system of claim 5, the control console being configured to receive the anatomical data manually.

7. The robotic system of claim 1, the control console being configured to update the instrument zone of operation based on feedback data.

8. The robotic system of claim 7, the instrument comprising at least one force sensor configured to generate the feedback data.

9. The robotic system of claim 1, the instrument comprising a memory configured to upload the instrument data to the control console wirelessly.

10. The robotic system of claim 1, the instrument comprising a switch configured to interact with the robotic arm in order to generate the instrument data.

11. The robotic system of claim 1, the control console comprising a control tower and a surgeon control console.

12. The robotic system of claim 1, further comprising a visual tracking system in communication with the control console, the visual tracking system being configured to track the position of the at least one external anatomical structure of the patient.

13. The robotic system of claim 12, the visual tracking system being configured to:
(i) spatially locate at least two fiducial bodies the at least one external anatomical structure of the patient and thereby track the position of the at least one external anatomical structure of the patient, and
(ii) communicate fiducial location data to the control console to thereby provide at least a portion of the patient tracking data.

14. The robotic system of claim 1, the robotic arm comprising a tool driver configured to longitudinally actuate the instrument.

15. The robotic system of claim 1, the instrument comprising a shaft, the shaft comprising a proximal linear portion and a distal curved portion.

16. A method of defining a zone of operation and limiting movement of at least a portion of an instrument within the zone of operation while the instrument is attached to a robotic arm, the method comprising:
receiving a set of data associated with either (i) at least one dimension of anatomy which the instrument is to be inserted into, or (ii) at least one dimension of the instrument;
calculating the zone of operation utilizing the set of data;
receiving instructions which instruct the robotic arm to actuate the portion of the instrument out of the zone of operation; and
filtering the received instructions such that the portion of the instrument remains within the zone of operation.

17. The method of claim 16, filtering the received instructions comprising ignoring the received instructions such that the instrument does not acuate in response to the received instructions.

18. The method of claim 16, filtering the received instructions comprising modifying the received instructions such that the instrument is actuated toward a boundary of the zone of operation.

19. The method of claim 16, the instrument comprising a uterine manipulator, calculating the zone of operation comprising utilizing anatomical data regarding a uterus.

20. A method of defining a zone of operation and limiting movement of a robotic arm within the zone of operation while the robotic arm is operatively coupled with an instrument, the method comprising:
receiving a set of patient tracking data from a patient tracking system, the set of patient tracking data indicating a position of at least one external anatomical structure of the patient;
calculating the zone of operation utilizing the set of patient tracking data;
receiving instructions that instruct the robotic arm to actuate out of the zone of operation; and
filtering the received instructions such that the robotic arm remains within the zone of operation.
